(19) United States
(12) Patent Application Publication
(10) Pub. No.: US 2011/0293484 A1
(43) Pub. Date: Dec. 1, 2011

(54) Apparatus for Drying and Sanitizing Items

(76) Inventors: Eric Stausgaard, Surrey (CA); Blake Drager, Surrey (CA)

(21) Appl. No.: 13/115,627

(22) Filed: May 25, 2011

Related U.S. Application Data
(60) Provisional application No. 61/348,810, filed on May 27, 2010.

(51) Int. Cl. A61L 9/12; B01J 19/08

(52) U.S. Cl. 422/116; 422/186.12

(57) ABSTRACT

An apparatus and a method for sanitizing air and equipment within a compartment includes an air moving device for drawing contaminated air from the compartment into a sanitizing unit in which the air is sanitized by generating ozone. The generated ozone is then removed with a catalyst which when irradiated by UV light creates oxidizers that react with the ozone and any remaining airborne contaminants providing additional air sanitization as well as ozone removal. The sanitized air is then returned into the compartment.

This patent application claims the benefit of U.S. provisional application No. 61/348,810 filed May 27, 2010. The disclosure of the provisional application is hereby incorporated herein by reference.

### TECHNICAL FIELD

The present invention relates to an apparatus and a method for drying items carried within a closed compartment, such as bag for carrying sports equipment, and more particularly, the present invention relates to an apparatus for sanitizing air and items within a compartment.

### BACKGROUND OF THE INVENTION

The use of ozone is well known as a disinfectant or sterilizing agent. Ozone (O₃) is an unstable gas comprising three atoms of oxygen. It is unstable because ozone gas will readily degrade back to its stable state, diatomic oxygen (O₂) with the formation of free oxygen atoms or free radicals. The free oxygen atoms are highly reactive and will oxidize almost everything (including viruses, fungi, moulds, bacteria, organic and inorganic compounds). The high level of the oxidation property of ozone means that in addition to being a disinfectant, ozone is capable of eliminating odors caused by animals, smoke and fuel. Following sanitization with ozone, the sanitized space will be left with a clean, fresh smell.

Penetrating properties of ozone as a gas in addition to its powerful oxidizing properties make ozone an ideal aerial disinfectant as well as a surface disinfectant of equipment, such as sports equipment, furniture, carpets, etc. However, the use of ozone for sanitizing purposes has drawbacks. Being a highly unstable and reactive form of oxygen, the ozone also reacts with living matter. Relatively low concentrations of ozone are known to cause headaches, nausea, and irritation of mucous membranes. Higher levels of ozone cause severe respiratory problems. Also if ozone directly contacts the equipment it may destroy or seriously damage the materials and/or fabrics from which the equipment is made. Consequently, ozone should, desirably, be removed from the air after generation.

Sports equipment is often comprised of many items for the individual user. These items form a bulky and unwieldy combination of items to move from place to place. Thus, sports equipment is usually transported in a bag.

Sports equipment has a reputation for becoming wet from sweat, and for becoming unpleasant-smelling as well. The unpleasant smelling sports equipment, and the space necessary to dry it, is currently an ongoing source of conflict in many household situations. Also, since many games are played soon after each other, a player often does not have enough time to properly dry the equipment before the next use. This makes for a clammy and unpleasant feeling; putting on wet or damp equipment. In addition, if not properly dried or cleaned, the sweat-soaked equipment becomes a site for growth of bacteria, mold, mildew, fungus, and other microorganisms that can spread disease, cause odor and/or damage or discolor the equipment.

Usually, damp garments, sport and athletic equipment, boots and shoes are removed from a sports bag, and spread out to dry out in storage areas having natural air circulation. When the equipment is dry, the equipment is placed back into the sports bag. For unpleasant odors, it is common practice to place aroma packs, air fresheners and the like into the sports equipment bag. However, if the sports equipment is not removed from the bag to dry, the effect of the air fresheners is greatly minimized.

Therefore, it is desirable to have an apparatus and/or method for drying, deodorizing, and/or sanitizing equipment, quickly and conveniently.

There are known solutions that have made an attempt to address this need. U.S. Pat. No. 3,432,939 describes a portable dryer with a blower to draw the air through a heating device that warms the air which is then circulated in the interior of the portable dryer, drying the items therein. While the portable dryer disclosed in U.S. Pat. No. 3,432,939 provides drying of the objects without removing them from the dryer, there is no sanitization of the items or the air within the dryer. G. L. Dhaemers in U.S. Pat. No. 5,369,892 discloses several self-contained dryers for accommodating articles, such as clothing, sporting equipment and shoes. The dryers have walls that cause heated air to re-circulate in the drying chambers to decrease the moisture content of the air and increase the energy efficiency of the drying. Blowers and fans associated with electric heaters operate to circulate heated air through the drying chambers. Ultraviolet lamps within the drying chamber destroy contaminants in the air and on the air conditioning coils, in the drying chamber. U.S. Pat. No. 5,369,892 does not describe any means for subsequently removing the generated ozone. Dhaemers in another U.S. Pat. No. 5,930,915 teaches a bag for carrying sports equipment with a blower that moves the air within the bag and through filters that are mounted on the bag to remove the odors and foreign matter from the air flow. No feature of disinfecting the items and air within the bag by generating ozone is taught by U.S. Pat. No. 5,930,915.

A similar configuration is taught by La Porte, U.S. Pat. No. 6,263,591 which describes a portable sports equipment drying container with a fan and a heating pad for circulating air through the container and drying moist or wet clothing.

A bag shown in U.S. Pat. No. 6,134,806 to Dhaemers discloses a bag with an air distributor connected with a hose to a blower and an ozone generator operable to move pressurized air and ozone into the air distributor. The air distributor moves the air and ozone into the bag to dry the sports equipment contained within the bag, to destroy bacteria, molds and fungus in the bag. The ozone directly contacts the sports equipment, which can be a serious problem because ozone can destroy many equipment materials. Also, ozone is a lung irritant and can leak out of the equipment bag and, dangerously, be inhaled, such as when the user opens the sports equipment bag.

U.S. Pat. No. 6,889,449 describes a cabinet for sanitizing sports equipment which comprises an ozone generator and a fan for circulating ozone containing gas through the cabinet. After the cabinet has been sanitized, the ozone is removed by spraying with an ozone depleting agent. The ozone also directly contacts the sports equipment, which may cause serious damage to the equipment materials.

Another apparatus for sanitizing and otherwise improving an atmosphere within a container is taught by Benedek and Carbone in the US pending patent No. 2010/0054989. Benedek and Carbone describe a bag in which the items within the bag are cleaned and sanitized by circulating cleaned and conditioned air across the materials. The contaminants that are transferred from the materials to the air are treated in an air cleaning unit. The air with the contaminants is drawn from the bag interior into the air cleaning unit where it first passes across an ozone generator zone in which the ozone is generated, then through the mixing zone for enhancing contact between the generated ozone and the contaminants. At the end the clean and ozone rich air is drawn through an ozone removal zone in which the ozone is destroyed. The bag described by Benedek and Carbone requires an air cleaning unit with three separate zones such as a zone for ozone generation, a zone for mixing ozone and contaminated air and a zone for ozone removal. Such cleaning unit is quite big in size, heavy and not suitable for portable bags.

Thus, there is a need for a compact, convenient, efficient and cost effective apparatus and method for drying, deodorizing and sanitizing air and equipment without damaging the equipment.

The foregoing examples of the prior art and the limitations related thereto are intended to be illustrative only and not exclusive. Other limitations of the prior art will become apparent upon reading the specification and study of the drawings.

### SUMMARY OF THE INVENTION

The present invention provides an apparatus for sanitizing air and equipment within a compartment comprising an air moving device for drawing contaminated air from the compartment into a sanitizing unit in which the air is sanitized by generating ozone. The generated ozone is simultaneously removed from the air and the sanitized air is returned into the compartment. A catalyst is also included which when irradiated by UV light creates hydroxyl radicals, super-oxide ions, hydroperoxides and/or any other oxidizers that react with the ozone and any remaining airborne contaminants providing additional air sanitization as well as ozone removal.

In one aspect, the present invention provides an apparatus for sanitizing air and items within a compartment comprising: a bag body having a plurality of walls comprising a bottom wall, a top wall and an openable panel, said walls and panel defining a bag interior space, wherein said panel provides access to the interior space; a sanitizing unit mounted on one of the walls for generating ozone and simultaneously removing ozone, said sanitizing unit comprising an air moving device for moving air in and out of said sanitizing unit; an inflow air channel for drawing air from the bag interior into the sanitizing unit, said inflow air channel having a first end at the top wall and a second end connected to said sanitizing unit; and an outflow air channel for expelling air into the bag interior, said outflow air channel having a first end connected to the sanitizing unit and a second end at the bottom wall.

In another aspect, the present invention provides an apparatus for drying items and sanitizing air comprising: a bag body having one or more walls and an openable cover panel, said one or more walls and panel defining a bag interior, wherein said panel provides access to the bag interior; a sanitizing unit mounted in the bag and having at least one ozone generator, at least one ozone remover and at least one air moving device; at least one airflow inlet in the bag interior for drawing air from the bag interior into said sanitizing unit; at least one air outlet for directing air out from the sanitizing unit and into the bag interior; and a plurality of locations for detachable moisture absorbing units to be positioned in the bag interior; wherein each said at least one ozone remover comprises a catalyst layer which when irradiated with light in an ultraviolet waveband creates oxidizers that react with ozone and air contaminants to produce sanitized air.

In yet another aspect the present invention provides an apparatus for sanitizing air and items comprising: a bag body having a bottom wall, a top wall, a cover panel, a back wall and two opposing side walls, said walls and panel defining a bag interior, wherein said cover panel provides access to the bag interior; a sanitizing unit mounted on one of the walls of the bag for generating ozone and simultaneously removing ozone from air, said sanitizing unit comprising one or more air moving devices for passing air through said sanitizing unit; at least one airflow inlet in the bag interior for drawing air from the bag interior into the sanitizing unit, said at least one airflow inlet providing communication between the bag interior and the sanitizing unit; and at least one outflow air channel formed in the bag interior for directing air into the bag interior, said at least one outflow air channel having a first end connected to the sanitizing unit and a second end at the bottom wall of the bag.

In addition to the exemplary aspects and embodiments described above, further aspects and embodiments will become apparent by reference to the drawings and study of the following detailed description.

### BRIEF DESCRIPTION OF THE DRAWINGS

FIG. 1 is a schematic side view of a bag with sanitizing unit according to an embodiment of the invention.

FIG. 2 is a schematic top view of a sanitizing unit according to an embodiment of the invention.

FIG. 3 is a perspective view of a bag according to an embodiment of the invention with a cover panel opened.

FIG. 4 is a schematic side view of a bag with a dual sanitizing unit according to an embodiment of the invention

FIG. 5 is a schematic top view of a dual sanitizing unit according to an embodiment of the invention.

### DETAILED DESCRIPTION OF EMBODIMENTS OF THE INVENTION

The figures and the following descriptions depict specific embodiments to teach those skilled in the art how to make and use the best mode of the invention. However, those skilled in the art would appreciate that the features described below can be combined in various ways to form multiple variations of the invention.

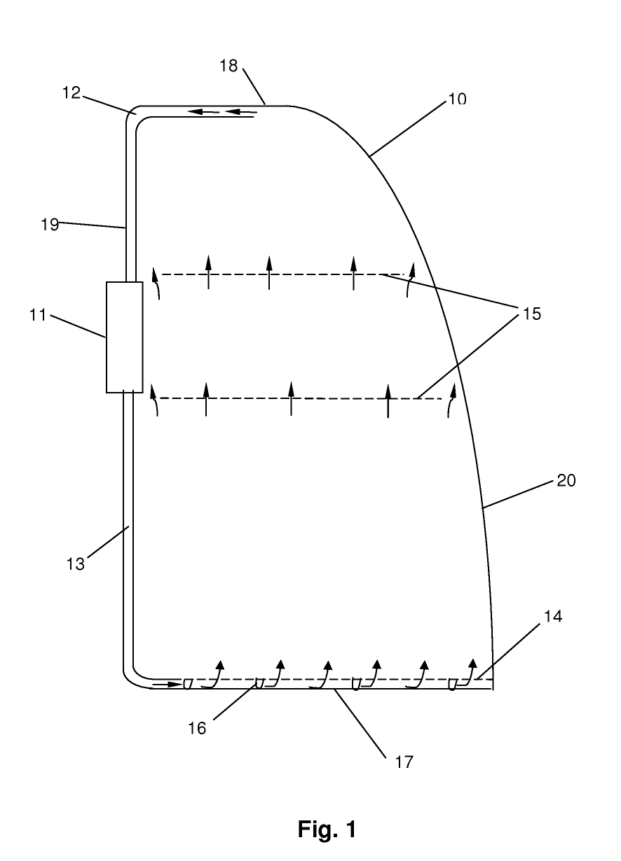

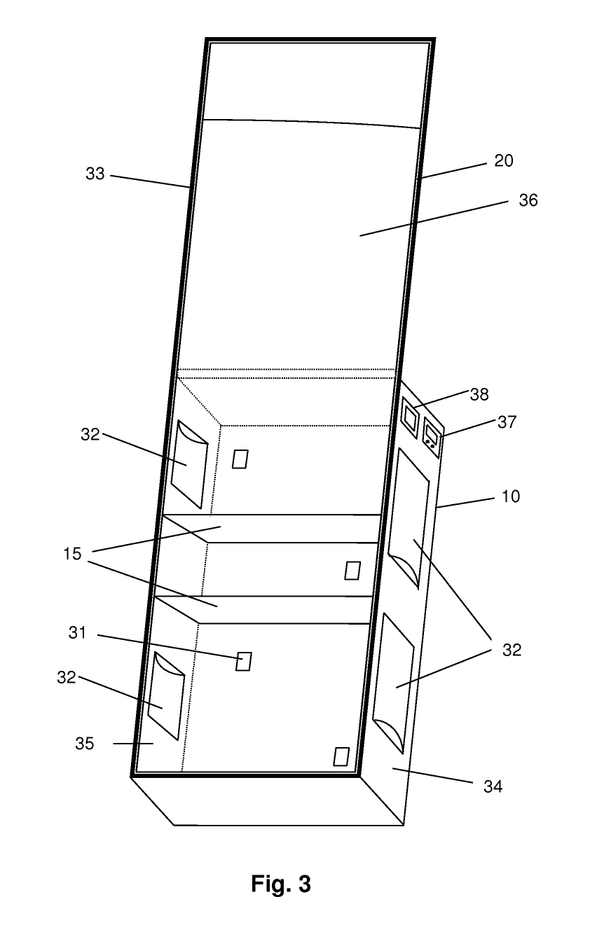

FIG. 1 illustrates a side view of an apparatus according to an embodiment of the invention. FIG. 1 shows a bag 10 which comprises a bag body with an approximately rectangular or polygonal cross section and six walls sewn together to define the bag body, namely: a bottom wall 17, a top wall 18, a back wall 19, two opposing side walls 34 and 35 (FIG. 3) and a cover panel 20. The bag may have a vertical or horizontal orientation. Persons skilled in the art would appreciate that the bag 10 can have any other shape and construction. The bag 10 is made of a rigid frame enclosed with some durable, flexible material. The cover panel 20 provides access to the interior of the bag for the insertion of clothes, equipment and any other items into the bag, and also to withdraw them from the interior of the bag. The cover panel 20 is attached to the top wall and is fastened to the bottom wall 17 and the two opposing side walls 34 and 35 with a zipper 33 (FIG. 3) which extends along the edges formed by the cover panel 20, the side walls and the bottom wall. However, any of the walls of the bag 10 may have zipper or any other releasable fastener that will allow equipment to be placed in and removed from the interior of the bag.

The bag 10 further includes a sanitizing unit 11, mounted on the back wall 19 of the bag, for cleaning the air drawn into the sanitizing unit 11 through an inflow air channel 12. The inflow air channel 12 is formed in the interior of the bag and is used to direct the air from the top of the bag into the sanitizing unit 11. The sanitized and clean air from the sanitizing unit flows through an outflow air channel 13 and is blown from the bottom of the bag into the bag interior. The inflow and outflow air channels 12 and 13 can also be formed on the exterior of the bag and have any cross-sectional shape and size, such as circular, rectangular or any other suitable shape or size which will permit the air to circulate from the bag interior through the sanitizing unit 11. The sanitizing unit 11 may be mounted on any wall of the bag 10.

In one embodiment, the sanitizing unit 11 may be detachable and can be used with various types of bags and containers.

The bag 10 also includes a floor panel 14 which is provided with a plurality of stub-like legs 16 for keeping the floor panel 14 spaced from the bottom wall 17. The floor panel 14 is meshed, i.e. it has a plurality of openings, which permit the sanitized air from the outflow air channel 13 to circulate into the interior of the bag. The bag 10 may also comprise one or more shelves 15 for organizing the items within the bag. If present, the one or more shelves 15 are also meshed to allow the air to move throughout the bag. Each of the shelves may be attached to the walls of the bag by means of VELCRO™ straps, or any other releasable fastener, which makes it convenient for the user to re-define the internal space of the bag to suit the user's personal organizational preferences. Of course, another embodiment may have fixed shelves, and/or fixed pockets or straps for holding skates or other items in position.

In another embodiment of the present invention, the bag 10 may include one or more separated hollow poles with a plurality of apertures therethrough that extend uprightly from the floor panel for supporting some parts of the equipment within the bag such as for example, shin guards, a helmet, skates, boots, gloves, pants, a chest protector etc. The items are positioned on the vertical poles so that when the sanitized air is blown from the bottom into the bag the sanitized air circulates across the items, sanitizing them by transferring the contaminants from the items into the airflow.

The bag 10 may also include a handle hingedly connected to an outer surface of one of the walls of the bag for carrying the bag. In addition the bag 10 may comprise integrated wheels and/or an integrated telescoping handle for wheeling the bag.

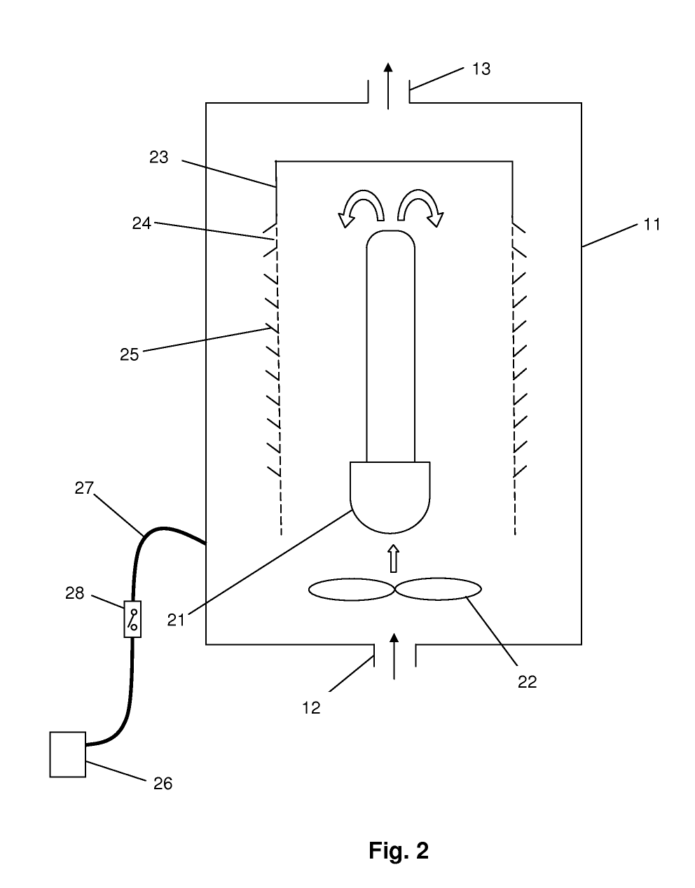

Details of the sanitizing unit 11 are shown in FIG. 2. It comprises an air moving device, such as a fan 22, which is used to draw air from the bag into the inflow air channel 12 and to discharge the air through the outflow air channel 13 into the interior of the bag. The fan 22 is a high flow fan which allows higher air flow to pass from the bag through the sanitizing unit 11 and back into the bag in a briefer time period than if a similar unit were used for sanitizing a room or other space occupied by people. The sanitizing unit 11 further has an ozone generating device 21 for generating ozone. The generated ozone as an unstable gas reacts with the contaminants from the air entering the sanitized unit 11, providing clean sanitized air. The ozone generating device 21 may comprise any source of ozone. For example, the ozone generating device 21 may be a UV light source, such as UV lamp or LED, which provides ultraviolet light at a wavelength suitable for generating ozone. A suitable wavelength may be in the UV-C range, and a range of wavelengths in the UV-C range may be emitted. The emission of the UV lamp may be centered around or have strong emission at the wavelengths at or near 185-187 nm, for example. In another embodiment, the ozone generating device 21 may be a corona discharge tube or any other suitable device that is capable of generating ozone.

After the ozone reacts with the contaminants in the airflow, the ozone is removed by an ozone removing device. In the embodiment shown in FIG. 2, the ozone removing device is a catalyst layer deposited onto inner and outer walls of a frame 23 that surrounds the UV lamp 21. The UV light from the UV lamp is incident on the catalyst layer, reacts with the catalyst and produces hydroxyl radicals, super-oxide ions, hydroperoxides and/or any other oxidizers. These oxidants are highly reactive and thus react with the ozone and with any contaminants left in the airflow which are not destroyed by the ozone. At least some of these oxidants pass into the volume of the bag, for further sanitizing the air and/or surfaces within it. The frame 23 has a plurality of openings 24 through which the air with the ozone passes, thus removing a significant amount of the generated ozone and any remaining contaminants. The ozone created within the sanitizer may be wholly or substantially destroyed before it leaves the sanitizer. The air passages 24 are relatively small which will result in a resistance to flow, but will also provide more reactive surface area and therefore more efficient ozone removal. The efficiency of the ozone removal is also increased by providing a plurality of flaps 25 which are positioned in proximity to the air passages 24. The flaps 25 are also coated with the catalyst layer. The flaps 25 are angled so to direct the airflow that passes throughout the air passages 24. As shown in FIG. 2, the flaps near the top of the frame 23 are angled in a direction opposite to the direction of the lower flaps. As a result the airflow that passes through the upper air passages will collide with the airflow that passes through the lower air passages creating air turbulence and providing additional time for the catalyst to remove the generated ozone from the air. In another embodiment, the orientation of the flaps 25 might be altered.

In one embodiment, the catalyst comprises a silica, a titanium oxide (TiO2), a manganese oxide (MnO2), a nano Nickel HCT™ catalyst, any combination of these or any other suitable metal oxide. In another embodiment, the catalysts can be carbon filters that may be heated. In another embodiment, the ozone can be wholly or partially removed by a UV light source that provides UV light at wavelength that provides decomposition of the ozone. This could be a wavelength or wavelength range in the UV-C band, such as 254 nm or a range containing this wavelength.

The sanitizer unit 11 also has reflective inner walls that reflect the UV that passes through the flaps back into the enclosure, and also onto the outer surfaces of the catalyst-coated frame 23. This increases the effectiveness of the UV that is produced by the lamp, and allows more oxidizing agents to be produced by the catalyst. The air in the bag circulates through the sanitizing unit until substantially all the contaminants and odor are removed. Since the airflow that is blown into the bag from the outflow air channel is substantially free of ozone, or at least the level of ozone is low there is no need of expensive monitoring means to monitor the amount of the ozone within the bag and also the equipment within the bag is not exposed to the ozone thus preventing any damage to it.

In one embodiment, the sanitizer unit 11 may be connected to a mains electric power supply 26 by an electric cord 27. A switch 28 for turning the sanitized unit on and off is also provided. The sanitizer unit 11 may also include a removable silicone skin wrapped around sanitizer's body for protecting it from any damage from dropping, smashing or any other shock.

In another embodiment the sanitizer unit 11 can be battery operated.

FIG. 3 shows a bag according to an embodiment of the invention with a cover panel opened. The cover panel 20 is folded back providing access to the interior of the bag 10. The cover panel 20 is provided with a zipper 33 for closing and opening the bag. Other suitable means for fastening the cover panel can be used for example, flexible straps with buckle at the end, or VELCRO straps or any other releasable fastener that can provide easy opening and closing of the cover panel 20. The bag 10 shown in FIG. 3 further comprises a plurality of moisture absorbing packs 31 for removing the moisture from the air and the items within the bag and drying them. The bag may also include one or more pockets 32 which may be located on the outside or inside surfaces of the walls.

The moisture absorbing packs 31 can be positioned anywhere within the interior of the bag. For example, the moisture absorbing packs 31 can be attached to any of the interior walls of the bag or can be placed within some parts of the equipment such as skates, or helmet, or shoes. The moisture absorbing packs 31 can also be provided within any of the additional pockets 32 of the bag 10.

In one embodiment the cover panel 20 contains a pocket 36 fitted on its inner surface provided for placing some parts of the equipment within it.

The moisture absorbing packs 31 are provided with a fastening means that allow the packs 31 to be easily repositioned or replaced. The moisture absorbing material used can be an activated alumina, a zeolite type dehydrator, an activated carbon or any other suitable moisture absorbent.

The quantity of the desiccant within the absorbing packs is such that the trapped moisture may be released into the atmosphere when the bag is opened, allowing it to be reused. The meshed shelves 15 accommodate a preferable set of contents and allow the airflow to move throughout the bag.

In one embodiment, the bag 10 may include control means, such as a timer 37, for setting the time period for sanitization.

In yet another embodiment, the bag 10 may comprises a lock and/or an alarm means 38 that may be turned on during the sanitizing time period so that the bag cannot be opened during the sanitizing period and cause unwanted harm from any residual ozone that might be present in the bag. After a predetermined time period the ozone generating device will be turned off and a control means will signal and/or activate to open any lock and/or to deactivate any alarm.

In operation, the equipment is placed in the bag 10. The moisture absorbing packs 31 are then positioned within the equipment and on the bag interior walls. The bag 10 is closed and the sanitizing unit 11 is plugged in and turned on by a switch. This will activate the fan 22 and the UV lamp 21 (FIG. 2) causing contaminated air from the top of the bag to be drawn through the inflow air channel 12 into the sanitized unit 11. The contaminated air passes the UV lamp 21 where the generated ozone reacts and neutralizes any airborne contaminants in the airflow. The sanitized air with the ozone passes through the apertures 24 formed in the frame 23 where the ozone and any remaining contaminants interact with the metal catalyst coated on the frame 23 providing sanitized air substantially free of ozone. The sanitized air through the outflow air channel 13, at the bottom of the bag 10, is blown into the interior of the bag. The sanitized airflow continues in its path through the meshed floor panel 14 and the meshed shelves 15 moving past the equipment and clothes therein and thus drying them and carrying out any contaminants therein. Such contaminated airflow is then again drawn through the inflow air channel 12 into the sanitizing unit 11.

After the pre-determined sanitizing time period expires, the control means 37 signals to the locking and alarm means 38 for unlocking the bag 10 and deactivating the alarm.

The apparatus subject to this invention can be used for sanitizing the air and the items in any kind of closed compartment such as any kind of storage or transportation compartment. It can also be used for sanitizing toys in hospitals or day care facilities or for sanitizing compartments for medical equipment or medical samples.

In another embodiment, the catalyst, bulb and/or airflow within the sanitizing unit 11 may be configured to allow some ozone to escape into the volume of the bag.

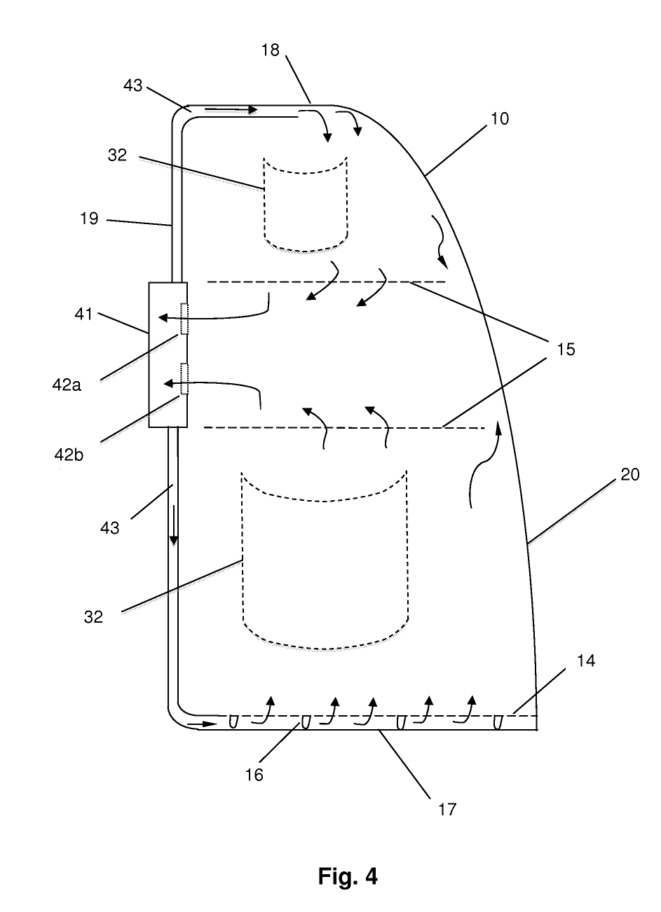

FIG. 4 illustrates a side view of an apparatus according to another embodiment of the invention. FIG. 4 shows a bag 10 having a bottom wall 17, a top wall 18, a back wall 19, two opposing side walls 34 and 35 (FIG. 3) and a cover panel 20. The bag may have a vertical or horizontal orientation. Persons skilled in the art would appreciate that the bag 10 can have any other shape.

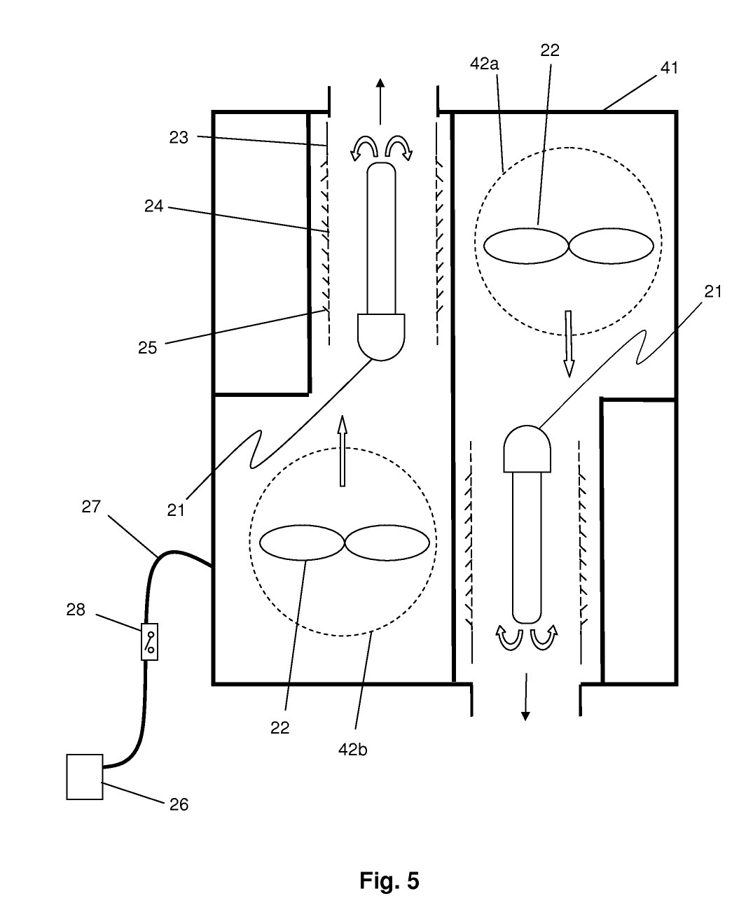

The bag 10 further includes a sanitizing unit 41, mounted on the back wall 19 of the bag, for cleaning the air drawn into the sanitizing unit 41 through dual airflow inlets 42a and 42b. The dual airflow inlets 42a and 42b are used to direct the air from the interior center of the bag into the sanitizing unit 41. The dual airflow inlets 42a and 42b can be covered with a protective vent cover as long as air can freely flow into the sanitizing unit 41. The sanitized and clean air from the sanitizing unit 41 flows through outflow air channels 43 and is blown from the bottom and top of the bag into the bag interior. Air flowing in through airflow inlet 42a into the sanitizing unit is sanitized and then clean air from the sanitizing unit 41 flows through an outflow air channel 43 and is blown from the bottom into the bag interior. Air flowing in through airflow inlet 42b into the sanitizing unit is sanitized and then clean air from the sanitizing unit 41 flows through an outflow air channel 43 and is blown from the top into the bag interior. While providing a crossover of airflow in this embodiment as seen in FIG. 5, the relative positions of airflow inlets 42a, 42b may be different in other embodiments. The dual outflow air channels 43 can be formed on the exterior or interior of the bag and have any cross-sectional shape and size, such as circular, rectangular or any other suitable shape or size which will permit the air to circulate to the bag interior from the sanitizing unit 41. The dual airflow inlets 42a and 42b may be connected to channels formed on the exterior or interior of the bag and the channels may have any cross-sectional shape and size, such as circular, rectangular or any other suitable shape or size which will permit the air to circulate from the bag interior into the sanitizing unit 41. The outflow air channels 43 can also have a plurality of openings to allow air flow into the interior of the bag. For example there could be small perforations along channels 43 with larger outlets at the bottom and top of the bag. The sanitizing unit 41 may be mounted on any wall of the container 10.

In one embodiment, the sanitizing unit 41 may be detachable and can be used with various types of bags and containers.

The bag 10 also includes a floor panel 14 which is provided with a plurality of stub-like legs 16 for keeping the floor panel 14 spaced from the bottom wall 17. The floor panel 14 is meshed, i.e. it has a plurality of openings, which permit the sanitized air from the outflow air channel 43 to circulate into the interior of the bag. The bag 10 may also comprise one or more shelves 15 for organizing the items within the bag. The one or more shelves 15 are also meshed to allow the air to move throughout the bag. Each of the shelves is attached to the walls of the bag by means of VELCRO™ straps, or any other releasable fastener, which makes it convenient for the user to redefine the internal space of the bag to suit its personal organizational preferences. Of course, another embodiment may have fixed shelves, and/or fixed pockets or straps for holding skates or other items in position.

In another embodiment of the present invention, the bag 10 may include one or more separated hollow poles with a plurality of apertures therethrough that extend uprightly from the floor panel for supporting some parts of the equipment within the bag such as for example, shin guards, a helmet, skates, boots, gloves, pants, a chest protector etc. The items are positioned on the vertical poles so that when the sanitized air is blown from the bottom into the bag the sanitized air circulates across the items, sanitizing them by transferring the contaminants from the items into the airflow.

The bag 10 may also include a handle hingedly connected to an outer surface of one of the walls of the bag for carrying the bag. In addition the bag 10 may comprise integrated wheels and/or an integrated telescoping handle for wheeling the bag.

Details of the sanitizing unit 41 are shown in FIG. 5. It comprises two air moving devices, such as a fan 22, which are used to draw air from the bag into the sanitizing unit 41 and to discharge the air through the outflow air channels 43 into the interior of the bag. The dual air moving devices, 22 allow for higher air flow to pass from the bag through the sanitizing unit 41 and back into the bag in a briefer time period than a unit with a singular air moving device. The sanitizing unit 41 further has dual ozone generating devices 21 for generating ozone. The generated ozone as an unstable gas reacts with the contaminants from the air entering the sanitized unit 41, providing clean sanitized air. The ozone generating device(s) 21 may comprise any source of ozone. For example, the ozone generating device(s) 21 may be a UV light source, such as UV lamp or LED, which provides ultraviolet light at a wavelength suitable for generating ozone. A suitable wavelength may be in the UV-C range, and a range of wavelengths in the UV-C range may be emitted. The emission of the UV lamp may be centered around or have strong emission at the wavelengths at or near 185-187 nm, for example. In another embodiment, the ozone generating device 21 may be a corona discharge tube or any other suitable device that is capable of generating ozone.

After the ozone reacts with the contaminants in the airflow, the ozone is removed by an ozone removing device. In the embodiment shown in FIG. 5, there are two ozone removing devices. They have a catalyst layer deposited onto inner and outer walls of frame(s) 23 that surrounds the UV lamp(s) 21. The UV light from the UV lamp is incident on the catalyst layer, reacts with the catalyst and produces hydroxyl radicals, super-oxide ions, hydroperoxides and/or any other oxidizers. These oxidants are highly reactive and thus react with the ozone and with any contaminants left in the airflow which are not destroyed by the ozone. At least some of these oxidants pass into the volume of the bag, for further sanitizing the air and/or surfaces within it. The frames 23 have a plurality of openings 24 through which the air with the ozone passes, thus removing a significant amount of the generated ozone and any remaining contaminants. The air passages 24 are relatively small which will result in a resistance to flow, but will also provide more reactive surface area and therefore more efficient ozone removal. The efficiency of the ozone removal is also increased by providing a plurality of flaps 25 which are positioned in proximity to the air passages 24. The flaps 25 are also coated with the catalyst layer. The flaps 25 are angled so to direct the airflow that passes throughout the air passages 24. As shown in FIGS. 2 and 5, the flaps near the top of frames 23 are angled in a direction opposite to the direction of the lower flaps. As a result the airflow that passes through the upper air passages will collide with the airflow that passes through the lower air passages creating air turbulence and providing additional time for the catalyst to remove the generated ozone from the air. In another embodiment, the orientation of the flaps 25 might be altered.

As described earlier in the specification, the catalyst may comprise a silica, a titanium oxide (TiO2), a manganese oxide (MnO2), a nano Nickel HCT™ catalyst, any combination of these or any other suitable metal oxide. In another embodiment, the catalysts can be carbon filters provided with heat. In another embodiment, the ozone can be wholly or partially removed by a UV light source that provides UV light at wavelength that provides decomposition of the ozone. This could be a wavelength or wavelength range in the UV-C band, such as 254 nm or a range containing this wavelength.

The sanitizer unit 41 also has reflective inner walls that reflect the UV that passes through the flaps back into the enclosure, and also onto the outer surfaces of the catalyst-coated frames 23. This increases the effectiveness of the UV that is produced by the lamps, and allows more oxidizing agents to be produced by the catalyst. The air in the bag circulates through the sanitizing unit until substantially all the contaminants and odor are removed. Since the airflow that is blown into the bag from the outflow air channels 43 is substantially free of ozone, or at least the level of ozone is low there is no need of expensive monitoring means to monitor the amount of the ozone within the bag and also the equipment within the bag is not exposed to the ozone thus preventing any damage to it.

In one embodiment, the sanitizer unit 41 may be connected to a mains electric power supply 26 by an electric cord 27. A switch 28 for turning the sanitized unit on and off is also provided. The sanitizer unit 41 may also include a removable silicone skin wrapped around the sanitizer's body for protecting it from any damage from dropping, smashing or any other shock.

In another embodiment the sanitizer unit 41 can be battery operated.

The bag 10 may also include one or more pockets 32 which may be located on the outside or inside surfaces of the walls.

The moisture absorbing packs 31 can be positioned anywhere within the interior of the bag. For example, the moisture absorbing packs 31 can be attached to any of the interior walls of the bag or can be placed within some parts of the equipment such as skates, or helmet, or shoes. The moisture absorbing packs 31 can also be provided within any of the additional pockets 32 of the bag 10.

In another embodiment, the catalyst(s), bulb(s) and/or airflow within the sanitizing unit 41 may be configured to allow some ozone to escape into the volume of the bag.

While preferred embodiments of the present invention are shown and described, it is envisioned that those skilled in the art may devise various modifications of the present invention without departing from its scope.

1. An apparatus for sanitizing air and items within a compartment comprising:
a bag body having a plurality of walls comprising a bottom wall, a top wall and an openable panel, said walls and panel defining a bag interior space, wherein said panel provides access to the interior space;
a sanitizing unit mounted on one of the walls for generating ozone and simultaneously removing ozone, said sanitizing unit comprising an air moving device for moving air in and out of said sanitizing unit;
an inflow air channel for drawing air from the bag interior into the sanitizing unit, said inflow air channel having a first end at the top wall and a second end connected to said sanitizing unit; and
an outflow air channel for expelling air into the bag interior, said outflow air channel having a first end connected to the sanitizing unit and a second end at the bottom wall.

2. An apparatus according to claim 1 further comprising a plurality of detachable moisture absorbing units positioned in the interior of the bag.

3. An apparatus according to claim 1 further comprising a meshed floor panel positioned at the bottom wall, said floor panel being separated from the bottom wall.

4. An apparatus according to claim 1 wherein the sanitizing unit further comprises a device for generating ozone to create ozone enriched air and a device for removing the ozone comprising a plurality of air passages coated with a catalyst through which the ozone enriched air passes.

5. An apparatus according to claim 4 wherein said plurality of air passages direct the air flow in opposing directions to create turbulence.

6. An apparatus according to claim 1 wherein said sanitizing unit further comprises a controller for operating said sanitizing unit for a predetermined time period.

7. An apparatus according to claim 1 further comprising one or more removable meshed shelves.

8. An apparatus according to claim 1, wherein said outflow air channel has more than one opening through which air is expelled into the bag interior.

9. An apparatus for drying items and sanitizing air comprising:
a bag body having one or more walls and an openable cover panel, said one or more walls and panel defining a bag interior, wherein said panel provides access to the bag interior;
a sanitizing unit mounted in the bag and having at least one ozone generator, at least one ozone remover and at least one air moving device;
at least one airflow inlet in the bag interior for drawing air from the bag interior into said sanitizing unit;
at least one air outlet for directing air out from the sanitizing unit and into the bag interior; and
a plurality of locations for detachable moisture absorbing units to be positioned in the bag interior;
wherein each said at least one ozone remover comprises a catalyst layer which when irradiated with light in an ultraviolet waveband creates oxidizers that react with ozone and air contaminants to produce sanitized air.

10. An apparatus according to claim 9 wherein said light creates the ozone at the same time as it creates the oxidizers.

11. An apparatus for sanitizing air and items comprising:
a bag body having a bottom wall, a top wall, a cover panel, a back wall and two opposing side walls, said walls and panel defining a bag interior, wherein said cover panel provides access to the bag interior;
a sanitizing unit mounted on one of the walls of the bag for generating ozone and simultaneously removing ozone from air, said sanitizing unit comprising one or more air moving devices for passing air through said sanitizing unit;
at least one airflow inlet in the bag interior for drawing air from the bag interior into the sanitizing unit, said at least one airflow inlet providing communication between the bag interior and the sanitizing unit; and
at least one outflow air channel formed in the bag interior for directing air into the bag interior, said at least one outflow air channel having a first end connected to the sanitizing unit and a second end at the bottom wall of the bag.

12. An apparatus according to claim 11, further comprising a second outflow air channel formed in the interior of the bag for directing air into the interior of the bag, said second outflow air channel having a first end connected to the sanitizing unit and a second end at the top wall of the bag.

13. An apparatus according to claim 11, wherein said at least one airflow inlet comprises at least two distinct and separate airflow inlets in the bag interior for drawing air from the bag interior into the sanitizing unit, said separate airflow inlets providing communication between the bag interior and the sanitizing unit.

14. An apparatus according to claim 11 further comprising a plurality of detachable moisture absorbing units positioned in the interior of the bag.

15. An apparatus according to claim 11 further comprising a meshed floor panel positioned at the bottom wall of the bag, said floor panel being separated from the bottom wall defining a space between the bottom wall and the floor panel.

16. An apparatus according to claim 11 wherein the sanitizing unit further comprises a device for generating ozone to create ozone enriched air and a device for removing the ozone comprising a plurality of air passages coated with a catalyst through which the ozone enriched air passes.

17. An apparatus according to claim 16 wherein said plurality of air passages direct the air flow in opposing directions to create turbulence.

18. An apparatus according to claim 11 wherein said sanitizing unit further comprises a controller for operating said sanitizing unit for a predetermined time period.

19. An apparatus according to claim 11, further comprising one or more removable meshed shelves.

20. An apparatus according to claim 11, wherein said at least one outflow air channel has more than one opening through which air can pass into the bag interior.
